(19) United States
(12) Patent Application Publication
(10) Pub. No.: US 2009/0304816 A1
(43) Pub. Date: Dec. 10, 2009

(54) METHOD AND COMPOSITION FOR TREATING AND DIAGNOSING RESTLESS LEGS SYNDROME

(75) Inventors: Ludger Grote, Goteborg (SE); Jan Hedner, Goteborg (SE); Kaj Stenlof, Torslanda (SE)

Correspondence Address: DICKSTEIN SHAPIRO LLP, 1633 Broadway, NEW YORK, NY, 10019, US

(73) Assignee: CEREUSCIENCE AB, Goteborg (SE)

(21) Appl. No.: 12/158,172

(22) PCT Filed: Dec. 6, 2006
(86) PCT No.: PCT/SE06/50553

§ 371 Date: Aug. 20, 2008

(30) Foreign Application Priority Data
Dec. 20, 2005 (SE) 0502830-3

(51) Int. Cl. A61K 31/137; A61K 31/195; A61K 33/26; A61K 31/404; A61K 31/437; A61K 31/428; A61K 31/48; A61K 31/4985

(52) U.S. Cl. 424/648; 514/567; 514/654; 514/418; 514/288; 514/367; 514/284; 514/250

(57) ABSTRACT

A method of treating Restless Legs Syndrome (RLS) comprises the joint administration of an agent selected from dopamine turnover increasing agent and dopaminergic receptor exciting agent, in particular pramipexole, and iron in a biologically usable form, in pharmacologically effective combined amounts. Also disclosed is a corresponding use; a pharmaceutical composition comprising an agent selected from dopamine turnover increasing agent and dopaminergic receptor exciting agent, in particular pramipexole, and iron in a biologically usable form, and a pharmaceutically acceptable carrier; a package comprising a pharmaceutical composition for per-oral administration comprising an agent selected from dopamine turnover increasing agent and dopaminergic receptor exciting agent and a pharmaceutically acceptable carrier and a pharmaceutical composition for per-oral administration comprising iron in a biologically usable form and a pharmaceutical acceptable carrier.

### FIELD OF THE INVENTION

The present invention relates to a method of treating and diagnosing restless legs syndrome including periodic limb movements during sleep and to a means for carrying out the method.

### BACKGROUND OF THE INVENTION

Patients with Restless Leg Syndrome (RLS) have difficulties to remain seated or even to stand still. Activities that require maintaining motor rest and include limited cognitive stimulation, such as transportation (travelling by car, plane, train, etc.) or attending longer meetings, lectures, movies or other performances, become difficult or even impossible. The symptoms typically worsen during the evening and early night period, a subgroup of RLS patients actually experience great difficulties to sleep and insomnia is frequently a prominent complication. The symptoms have a considerable negative impact on quality of life. They can typically be relieved by movement, such as standing up, moving around, or short walks. However, the symptoms may return with increased intensity shortly after such activities. If an RLS patient is forced to lay still, symptoms will continue and may led to involuntary movements.

The majority of RLS patients exhibit periodic limb movements during sleep (PLMS) or periodic limb movements during wakefulness (PLMW). PLMS are best described as rhythmic extensions of the foot, big toe and dorsal flexions of the ankle.

Occasionally, this movement is accompanied by flexion of the knee and hip. The movements last for approximately 0.5 to 5 seconds and appear with a frequency of about one every 20 to 40 seconds. PLMS occur in cluster episodes, each of which lasts several minutes or even hours. PLMS/PLMW and RLS may be found independently from each other but epidemiological data suggests that approximately 90% of RLS patients also have considerable periods of PLMS. However, PLMS may occur in patients without RLS symptoms during wakefulness.

The clinical diagnosis of RLS is based on four major criteria which should be met (See: Sleep Med. 2003 Mar; 4 (2):121-32, Walters A S, LeBrocq C, Dhar A, Hening W, Rosen R, Allen R P, Trenkwalder C; International Restless Legs Syndrome Study Group. Validation of the International Restless Legs Syndrome Study Group rating scale for restless legs syndrome). These include: (1) A sensation of an urge to move the limbs (usually the legs, but also arms or the trunk may be involved); (2) motor restlessness to reduce sensations; (3) when at rest, symptoms often return or get worse; and (4) there is a marked circadian variation with a peak occurrence or severity of RLS symptoms during evening and early night.

RLS and PLMS are typically diagnosed by patient history and standardized questionnaires as well as by polysomnographic evaluation. A ten-question evaluation scale developed by the International RLS study group (IRLSSG) has been found to be useful for assessment of RLS severity for purposes of clinical assessment, research, or therapeutic trials. Standardized tests such as the Suggested Immobilization Test and the Forced Immobilization Test for quantification of RLS or PLM have been proposed.

A number of studies suggest that the fundamental pathophysiology of RLS/PLMS involves mechanisms of iron and dopamine transport and turn-over. Reduced iron content of the brain and other fluids/compartments of the body as well as reduced dopamine synthesis in the brain have been proposed in RLS. Dopamine is a neuro-transmitter synthesized in the brain and with essential features for adequate central nervous system (CNS) function. Iron is a cofactor for the enzyme tyrosine hydroxylase which is the rate-limiting step in dopamine metabolism. In addition, experimental data points to iron as an essential component for adequate transmembraneous transport of dopamine and dopamine receptor function in CNS regions responsible for motor and sensory function. Iron deficiency, by its potential effects on dopamine system activity has been identified as an important component in RLS pathophysiology.

Reduced iron content and availability leads to an impairment of dopamine availability as a result of reduced tyrosine hydroxylase activity or other mechanisms intimately involved in dopamine synthesis and metabolism. Animal experiments demonstrated that substances that bind metals such as iron, thereby reducing physiological availability of said metal, were effective in reducing dopamine and dopamine-turnover. In iron-deficient animals, dopamine receptors, dopamine transporter function and receptor density were impaired while extracellular dopamine was elevated. Along these lines there are observations in RLS patients showing a decrease in dopamine receptor content in basal ganglia, a 65% reduction of cerebral spinal fluid (CSF) ferritin and a three-fold increased CSF transferrin (iron transport protein in blood and body fluids) concentration, despite normal serum levels of ferritin and transferrin. These findings strengthen the hypothesis that both iron and dopamine deficits, particularly at the level of the central nervous system, play an essential role in the occurrence of RLS. Although less extensively investigated, there is a consensus that the principles of RLS pathophysiology may be extended to conditions in PLMS/PLMW and they are in principle analogous to those in RLS. Impairment of sleep by frequent awakenings and associated consequences for daytime function and quality of life are important features in this condition. In this application the conditions of RLS and PLMS/PLMW are jointly referred to as RLS.

A number of different treatment modalities are currently available in RLS. These include the administration of dopamine receptor agonists, other dopaminergic agents, benzodiazepines, opiates and anti-convulsants. However, the use of several of these agents is hampered by undesirable side effects that, depending on the substance, include nausea, vomiting, insomnia, daytime sedation, cognitive side effects, allergic reactions, anaphylactic shock etc. Certain forms of RLS, so called secondary RLS a condition that is related to e.g. pregnancy or end-stage renal disease, may be specifically resolved be treatment or elimination of the underlying condition/disease. In these cases there may a profound reduction or even complete remission of RLS following treatment.

Intake of oral levodopa generally treats RLS effectively during the first weeks or months of treatment. However, continued use frequently leads to tolerance development, augmentation of symptoms or even a general worsening of RLS. Similar effects are frequently seen during long-term treatment with dopamine receptor agonists. Other frequently used remedies like benzodiazepines, opiates and anti-convulsants are uniformly less effective than the dopamine agents and side effects are prevalent in a manner that clearly limits their clinical applicability.

### OBJECTS OF THE INVENTION

As evident from the preceding description of the state of the art, there is a need for an improved method of treating RLS. In particular, a new pharmacological treatment would offer a definite advantage in front of the methods used at present, many of which provide insufficient relief and some of which are associated with potentially severe side effects and limitations.

One object of the present invention is thus to provide a method for the treatment of RLS, which reduces and/or eliminates some or all of the drawbacks of the methods known in art. Another object of the invention is to provide a means for carrying out said method.

A further object of the present invention is to provide a diagnostic tool for detection the presence of RLS/PLMW and PLMS in a patient and a corresponding diagnostic method.

Further objects of the invention will be evident from the following summary of the invention, a number of preferred embodiments illustrated in a drawing, and the appended claims.

### SUMMARY OF THE INVENTION

According to the present invention is provided a method of treating RLS including PLMS and PLMW, the method comprising the joint administration of an agent selected from dopamine turnover increasing agent and dopaminergic receptor exciting agent, and iron in a biologically usable form, in pharmacologically effective combined amounts. Surprisingly the administration of these combined amounts is more effective than the separate unrelated administration of a corresponding amount of the agent selected from dopamine turnover increasing agent and dopaminergic receptor exciting agent and a corresponding amount of iron in a biologically usable form. In the joint administration of the invention iron in a biologically usable form advantageously enhances the RLS dampening effect of the agent selected from dopamine turnover increasing agent and dopaminergic receptor exciting agent. A pharmacologically effective amount of the agent selected from dopamine turnover increasing agent and dopaminergic receptor exciting agent or a combination of several of such agents an is one which eliminates or substantially reduces or dampens the manifestations of RLS over a period of time, such as during the afternoon, evening, and even during nocturnal or other sleep periods of from 10 minutes to 10 hours.

In this application the agent selected from dopamine turnover increasing agent and dopaminergic receptor exciting agent is also referred to as “dopaminergic agent” or “DA agent”. Furthermore, in this application iron in a biologically usable form is referred to as “IR”. “Biologically usable form” relates to a form in which the iron can be taken up by the gastrointestinal mucosa tract or which is used by the body for restoring depleted iron stores upon injection or infusion. In this application the combination of the agent selected from dopamine turnover increasing agent and dopaminergic receptor exciting agent, and of iron in a biologically usable form is termed “DA agent/IR”. “Joint administration” indicates administration in a time-wise defined manner, either simultaneously or about simultaneously, or consecutive. “Joint administration” includes administration of the components of DA agent/IR in separate overlapping administration schemes.

Dopamine has been used for decades to treat a number of conditions including RLS.

Other recognized and documented indications for dopamine include Morbus Parkinson (cerebral D2 and D3 receptors), heart failure and cardiogenic shock (vascular D1 receptors). For a recent survey in respect of known therapeutic uses of dopamine, see: Goodman and Gilman's The Pharmacological Basis of Therapeutics, 10th Ed., Pergamon Press, New York etc., 2001.

Many agents with an excitatory effect on dopamine receptors are known in the art.

Their chemical structure varies considerably. Dopamine and central nervous dopaminergic effect promoting agents particularly useful in the invention include carbidopa and levodopa, dopamine, dobutamine, dopamine agonists like ropinerol, cabergoline, pramipexole, pergolide, rotigotine, lisuride and bromocriptine, as well as dopamine promoting MAO-B inhibitors like e.g. selegiline, rasagiline and safmamide, and dopamine reuptake inhibitors like e.g. vanoxerine (GBR 12909), radafaxine and SEP 226 330, including pharmaceutically acceptable salts, enantiomers of those in the aforementioned compounds which are able to form salt with organic or inorganic acids. The aforementioned compounds are extensively described in the literature; see, for instance: Goodman and Gilman's The Pharmacological Basis of Therapeutics, 10th Ed., Pergamon Press, New York, 2001 and Martindale The Complete Drug Reference 34th Ed., Pharmaceutical Press, New York, 2005 and the references cited therein. In this publication, which is hereby incorporated by reference, pharmaceutical compositions useful in the invention are described for a number of DA agents. All different chemical structures and specifically salts being only slightly soluble in aqueous solutions are included in the invention. This is specifically true for those chemical structures which may be of particular interest in the manufacture of controlled release DA agent/IR compositions. A potential DA agent/IR mixture is advantageously formulated in a way appropriate to the chosen administration route.

The positive effect of IR in the treatment of RLS may be due to an enhancement of dopaminergic activity in the central nervous system thereby mimicking the effects of dopamine described above. While this hypothesis may provide a scientifically attractive explanation for the observed effect of said IR, it must be emphasized that this must not be considered to be binding in any way on the concept and the working of the present invention. The IR in a biologically usable form of the invention is preferably a salt of Fe²⁺, with an acid, more preferred with an organic acid or a hydroxide of Fe²⁺. Preferred organic acids comprise ascorbic, aspartic, fumaric, gluconic, and succinic acid. Preferred inorganic acids comprise hydrochloric acid and sulphuric acid. The IR of the invention may be stabilised by complex formation such as with dextran, sorbitol, and sucrose. IR particularly useful in the invention includes ferrous fumarate, ferrous sulphate, ferrous gluconate, sodium ferrous gluconate, carbohydrate complexes of Fe++, such as iron dextran, iron sorbitol, iron sucrose in form of capsules, extended-release capsules, solutions, lozenges, syrups, suspensions, tablets, including chewable tablets, for per-oral administration, and aqueous solutions for parenteral administration. Preferred for intra-muscular injection is iron sorbitol, iron sucrose, and iron dextran in an aqueous carrier.

The DA agent/IR combination of the invention, which may comprise a mixture of several DA combined with an IR or several IR combined with a DA or several DA combined with several IR, can be administered by various routes. The most preferred route is by per-oral administration, in which case the pharmaceutical preparation In this context the agent of the invention may be designed for preferred uptake through the oral mucosa, such as by sub-lingual uptake. Also preferred is a preparation that releases the DA/IR agent of the invention so as to obtain essentially gastrointestinal absorption. Knowledge about clinical pharmacokinetics of DA and IR (see, for instance: Goodman and Gilman's The Pharmacological Basis of Therapeutics, 10th Ed., Pergamon Press, New York etc., 2001) is useful in designing the aforementioned pharmaceutical preparations. According to a preferred aspect of the invention the per-oral or parenteral administration of the DA agent and the IR is by separate pharmaceutical preparations which may be administered simultaneously or within a short period of time, such as within one or five or ten minutes or consecutively in intervals of up to 30 min or 2 hrs and even 12 or 24 hrs and more.

For preparing the aforementioned preparations formulation techniques known in the art may be used; in this context reference is made to Pharmaceutical Dosage Forms: Tablets. Vol. 1-3, H A Lieberman et al., Eds. Marcel Dekker, New York and Basel, 1998, which is hereby incorporated by reference. Specific reference is made to chapter 7 (Special Tablets, by J W Conine and M J Pikal), chapter 8 (Chewable Tablets, by R W Mendes, O A Anaebonam and J B Daruwala), and chapter 9 (Medicated Lozenges; by D Peters).

According to a second preferred aspect of the invention is disclosed a pharmaceutical composition comprising an agent selected from dopamine turnover increasing agent and dopaminergic receptor exciting agent, and iron in a biologically usable form, in a combined amount pharmacologically effective in the treatment of RLS.

According to third preferred aspect of the invention is disclosed a combination package comprising an IR preparation for intravenous infusion, in particular for a number of scheduled separate infusions to be administered during one or several weeks or even one month or more, and several dosages of a per-oral preparation comprising a DA agent for repeated administration over a period of time identical or overlapping the period of infusion of the IR preparation, such as on a daily or weekly basis. The dosages applied in such combination packages will be based on efficacy data routinely determined by clinical studies (e.g. repeated IR infusions of 1 g of iron sucrose in total for a three months period in combination with a once daily intake of 0.35 mg pramipexole. Another example of a combination package of the invention for per-oral administration of DA and IR comprises a tablet comprising 100 mg of ferrous sulphate in combination with a tablet comprising 0.35 mg of pramipexole.

In the event that a DA agent with a short pharmacological half-life is used, it is desirable to design an oral, buccal or sublingual pharmaceutical formulation for sustained release of the DA agent/IR combination of the invention to avoid the need af frequent administration which would be particularly difficult during sleep. A suitable solution for this problem would be a fixation, at least for a certain period of time, of one or both components of the formulation containing the DA agent/IR combination in or near the sublingual region. This could be done by a device for fixation or a holding the tablet, lozenge, or similar attached to one or several teeth of the lower jaw, or by implantation of a holding means, of titanium, for instance, in the lower jaw. Such holding means could also be used for holding a small plastic container enclosing a liquid or solid pharmaceutical composition of the DA of the invention, from which container the solution would leak through a minute opening or through a system of micropores driven by, for example, osmotic pressure. It is also possible to incorporate the compound of the invention in polymer matrix, biodegradable or not, from which it could leak slowly into the oral cavity. Appropriate technology for producing biodegradable polyester matrices of the polylactide/polyglycolide type for incorporation and sustained release of pharmacologically active compounds is described in, for instance, L A Sanders et al., J Pharmaceutical Sci. 75 (1986) 356-360, and in the U.S. Pat. No. 3,773,919 (Boswell). Non-degradable polymers of appropriate physical properties can also be used as matrices.

The amount of DA agent and IR to be administered in combination for treatment of RLS will vary depending on factors such as the particular chemical nature of the DA agent/IR formulation used, the route of administration, the release profile of the formulation into which it is incorporated, the severity of the disease, individual pharmacokinetic and pharmacodynamic properties as well as the status of the patient. For instance, the dose range for per-oral administration of pramipexole will be from 0.009 to 1 mg per 24 hours. Normally, an amount of from 0.18 to 0.5 mg of pramipexole is envisaged as the normal range used for a per oral administration to an adult person.

The dosage range for an IR preparation like iron sucrose may vary between 200 and 2000 mg. The appropriate dose range for a particular DA agent or IR or the combination of a DA agent and an IR can be determined by titration in routine experiments.

In addition to the methods of administration of the DA agent and IR of the invention mentioned above also parenteral, intranasal, and rectal administration is useful.

According to the invention the DA agent can be efficiently administered also by inhalation, such as inhalation via the mouth or via the nose. The nasal mucosa is easily accessible by use of extra- or intranasal devices, the later ones appropriately shaped and designed similarly to what has been described above for intraoral and sublingual administration. The transdermal formulation comprising the DA agent of the invention is specifically advantageous in regard of simplicity and from a patient comfort standpoint. In this case, the agent is applied to the skin in form of a viscous ointment or similar. Transdermal systems (patches provided with a liquid or semi-liquid pharmaceutical composition) for controlled drug delivery through the skin are well known in the art, for instance formulations used for administration of nicotine and drugs used for diseases of the circulatory system.

The timing of the administration of the composition and/or device comprising the DA agent/IR combination of the invention will depend on the particular compound, its rate of absorption through the mucosa or the skin, the release profile of the respective sustained release formulation and/or device, if used, and similar. Typically, administration of the DA agent/IR combination will, in the majority of cases, have to start well in advance of the RLS symptoms period to achieve optimal effect, for instance from 10 minutes to 6 hours prior to the onset of sleep.

The DA agent/IR combination of the invention may also be combined, in one and the same pharmaceutical preparation, with other pharmacologically active compounds useful in the treatment of RLS/PLMS.

The DA agent/IR combination of the invention may also be used for diagnosing RLS and thereby to dissociate this condition from other types of sleep disorders. The diagnostic method according to the invention comprises administration to the patient a DA agent/IR combination given in increasing amounts prior to or during a series of day/evening/sleep periods; administration can be in single or multiple doses. The observation of a reduction of the severity and/or RLS events or episodes or reduced daytime sleepiness/increased alertness is indicative of the presence of RLS.

The invention will now be explained in more detail by reference to a preferred but not limiting embodiment illustrated in a drawing showing the combined effect of DA and IR on clinical symptoms of RLS assessed by the International Restless Legs Syndrome Scale (IRLSS) in each of the patients.

### DESCRIPTION OF THE FIGURES

FIG. 1 is a diagram illustrating the clinical evaluation of two patients with RLS upon administration of iron sucrose and pramipexole;

FIG. 2 is a diagram illustrating the clinical evaluation of a third patient with RLS upon administration of a fixed combination of L-dopa and carbidopa, and iron dextran.

### DESCRIPTION OF A PREFERRED EMBODIMENT

### Example 1

### Single-Blind, Uncontrolled Treatment Studies With DA and Iron Sucrose in Three Different Patients With Restless Legs

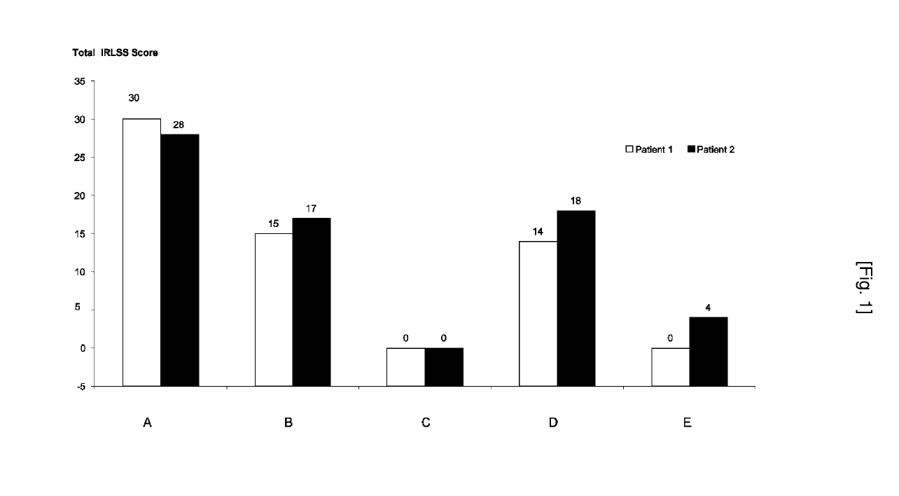

Two patients with moderate to severe RLS/PMLS (PLM index (PLMI) 3 and 17, IRLSS Score 30 and 28 respectively (FIG. 1; A), at baseline) were studied. Pramipexole (in form of the dihydrochloride monohydrate) 0.35 mg given once daily by an evening dose for 21 days, resulted in a mean reduction of PLMI from 3 to 0, and from 17 to 2,and the IRLSS score was reduced from 30 at baseline to 15 at day 21 and from 28 to 17, respectively (FIG. 1; B). No side effects were reported during the study from neither of the patients. Due to remaining RLS complaints iron sucrose therapy in the dosage 500 mg i.v. twice, was introduced during one week (FIG. 1; C). Both patients were free of any RLS complaints when assessments were performed 3 weeks after the last infusion of iron sucrose. Serum ferritin levels increased from 30/45 mg/dl at baseline to 130/145 mg/dl after iron sucrose infusions. Patients discontinued pramipexole treatment for one week and symptoms reoccurred (IRLSS scale after one treatment pause of pramipexole 14 and 18. FIG. 1; D). However, the baseline values of 30 and 28 in the IRLSS scale were not reached. Following reintroduction of pramipexole in the previously used dosages, the IRLSS score after 12 weeks was found to be 0 and 4in the two patients (FIG. 1; E).

This case report clearly demonstrates a potent reduction of PLMI and RLS complaints by a combination therapy of iron supplementation and dopaminergic agents. There was a clear additive effect of the two treatments with regard to control of RLS and PLM complaints emphasizing that the combination of drugs, DA and iron, results in an effect superior to that obtained by either drug used alone.

### Example 2

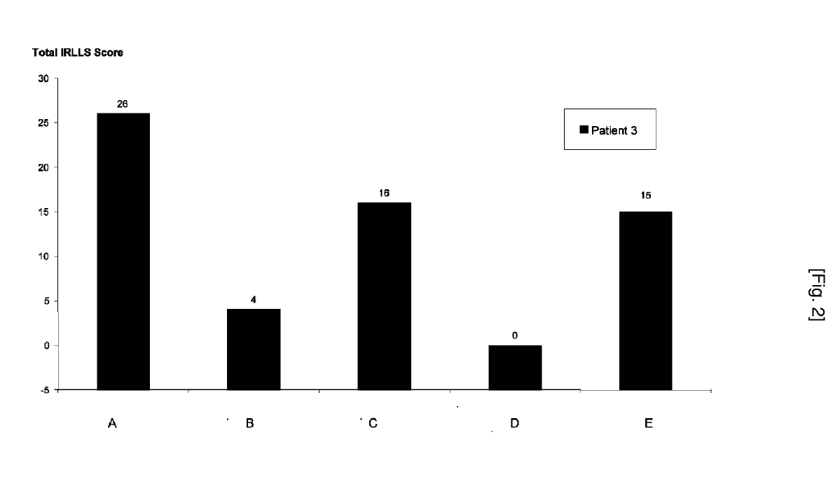

A subsequent clinical observational study included a patient with clinical symptoms of RLS and an IRLSS score of 26 on the diagnostic evaluation under treatment with L-dopa (levodopa) and carbidopa (Sinernet®, fixed combination dosage of 100 mg L-dopa and 25 mg carbidopa) (FIG. 2; A). The patient needed to be treated with altogether three tablets per evening in order to obtain acceptable symptom relief (IRLSS score 4. FIG. 2; B). However, this patient complained of considerable gastrointestinal side effects including nausea and vomiting when the treatment was optimized. In addition, following two months of treatment this patient had started to suffer from significant symptoms suggestive of augmentation evidenced by an onset of RLS symptoms in the early afternoon. Although reduction of the dosage to one tablet per evening abolished the side effects, and in part of the augmentation problem, the symptoms of RLS were not sufficiently controlled (IRLSS score 16. FIG. 2; C). Consequently, we administered orally to this patient, who had a well balanced iron blood status (serum ferritin 85 mg/dl), 200 mg iron dextran daily over 6 months. There was a considerable improvement and the patient demonstrated a complete remission from RLS (IRLSS score 0; FIG. 2; D) and no further augmentation problem. In addition, it was found that the patient could be treated continuously with a reduced dose of dopamine, one tablet per evening, with a continued relief from symptoms (IRLSS 0). An attempt to discontinue dopaminergic treatment altogether failed. Following this discontinuation there was a substantial symptom relapse (IRLSS score 15. FIG. 2; E). The treatment with iron dextran did not cause any adverse effects in this patient.

These case reports clearly demonstrate that the combination of iron treatment together with dopaminergic treatment of RLS may be used to reach improved RLS symptom control and that the dosage of dopaminergic agents may be reduced when used in combination relative to the use of DA alone as a single agent. The treatment based on a combined DA and iron treatment in RLS also resulted in a better RLS symptom control together with a lower frequency and severity of adverse treatment effects. In addition there was an improved capacity to control previous augmentation observed with the use of a single DA treatment.

1. A method of treating Restless Legs Syndrome (RLS) comprising the joint administration of an agent selected from dopamine turnover increasing agent and dopaminergic receptor exciting agent, and iron in a biologically usable form, in pharmacologically effective combined amounts.

2. The method of claim 1, wherein dopamine turnover increasing agent and dopaminergic receptor exciting agent is selected from the group consisting of levodopa, carbidopa, dopamine, dobutamine, dopamine agonist dopamine promoting MAO-B inhibitor and dopamine reuptake inhibitor and pharmaceutically acceptable salts of those in the aforementioned compounds capable of forming such salts.

3 The method of claim 1, wherein iron in a biologically usable form comprises ferrous ion in form of a salt or a hydroxide.

4. The method of claim 3, wherein the iron is complexed.

5. The method of claim 4, wherein the iron complexing agent comprises a carbohydrate.

6. The method of claim 5, wherein the carbohydrate is selected from the group consisting of dextran, sorbitol, and sucrose.

7. The method of claim 2, wherein the salt is a salt of an inorganic acid.

8. The method of claim 7, wherein the salt is chloride or sulphate.

9. The method of claim 2, wherein the salt is a salt of an organic acid.

10. The method of claim 9, wherein the salt is selected from the group consisting of ferrous fumarate, ferrous sulphate, ferrous gluconate, sodium ferrous gluconate, and ferrous adipate.

11. The method of claim 3, wherein the iron oxide is ferrous oxide.

12. The method of claim 1, wherein said joint administration is essentially simultaneous.

13. The method of claim 1, wherein said joint administration is consecutive.

14. The method of claim 1, wherein the administration period of the agent selected from dopamine turnover increasing agent and dopaminergic receptor exciting agent, and the administration period of iron in a biologically usable form overlap.

15. The method of claim 1, wherein administration starts from 10 min to 10 hrs prior to a sleep period.

16. The method of claim 1, wherein administration is per-oral parenteral or both.

17. The method of claim 16, wherein administration of the agent selected from dopamine turnover increasing agent and dopaminergic receptor exciting agent is per-oral and administration of iron in a biologically usable form is intramuscular or parenteral.

18. The method of claim 1 in which the agent selected of dopamine turnover increasing agent and dopaminergic receptor exciting agent are administered in a composition for sustained release.

19. The method of claim 1, wherein IR is administered in a dose of from 0.1 mg to 2500 mg.

20. (canceled)

21. The method of claim 2, wherein the dopamine agonist is selected from the group consisting of ropinerol, cabergoline, pramipexole, pergolide, bromocriptine, rotigotine and lisuride, the dopamine promoting MAO-B inhibitor is selected from the group consisting of selegiline, rasagiline and safinamide, and the dopamine reuptake inhibitor is selected from the group consisting of vanoxerine (GBR 12909), radafaxine and SEP 226 330.

22-24. (canceled)

25. A pharmaceutical composition for per-oral administration comprising an agent selected from dopamine turnover increasing agent and dopaminergic receptor exciting agent, iron in a biologically usable form, and a pharmaceutically acceptable carrier.

26. The composition of claim 25 in form of a tablet, lozenge, or capsule.

27. A package comprising a pharmaceutical composition for per-oral administration comprising an agent selected from dopamine turnover increasing agent and dopaminergic receptor exciting agent and a pharmaceutically acceptable carrier and a pharmaceutical composition for per-oral administration comprising iron in a biologically usable form and a pharmaceutical acceptable carrier.

28. The package of claim 27, wherein the agent selected from dopamine turnover increasing agent and dopaminergic receptor exciting agent is selected from the group consisting of levodopa, carbidopa, dopamine, dobutamine, dopamine agonist MAO-B inhibitor, and dopamine reuptake inhibitor, including pharmaceutically acceptable salts of those in the aforementioned compounds capable of forming such salts.

29. The package of claim 28, wherein iron in biologically usable form is in form of a ferrous salt of an inorganic or organic acid or in form of a ferrous oxide, optionally complexed by a carbohydrate.
